(19) United States
(12) Patent Application Publication
(10) Pub. No.: US 2007/0037285 A1
(43) Pub. Date: Feb. 15, 2007

(54) Method and supply unit for monitoring changes and states in reaction chambers

(76) Inventors: Ralf Ehret, Rostock (DE); Axel Kob, Rostock (DE); Elke Thedinga, Rostock (DE); Heiko Holst, Gross Labenz (DE)

Correspondence Address: JORDAN AND HAMBURG LLP, 122 EAST 42ND STREET, SUITE 4000, NEW YORK, NY, 10168, US

(21) Appl. No.: 10/574,338

(22) PCT Filed: Oct. 1, 2004
(86) PCT No.: PCT/EP04/52405

§ 371 Date: May 8, 2006

(30) Foreign Application Priority Data
Oct. 3, 2003 (DE) DE 10346451.4

(51) Int. Cl. G01N 33/00

(52) U.S. Cl. 436/34; 422/68.1

(57) ABSTRACT

The invention relates to a method for monitoring changes and states in reaction chambers as well as a supply unit which is used for introducing a liquid culture medium during cell culture analyses. The aim of the invention is to allow for an air bubble-free measurement in reaction chambers so as to monitor changes and states therein without using any degassing process. Said aim is achieved by withdrawing or pumping off a fluid from a receptacle via a hose and/or pipe system and conveying said fluid to a supply unit. The fluid drips or flows into a trickling chamber via an inlet duct, said fluid forming a supply above a head and a reaction chamber. The height of the fluid level, and thus the supply volume, is determined with the aid of a suction duct.

### BACKGROUND OF THE INVENTION

Experiments on cell cultures are performed in reaction chambers. Cells, cell components, DNA, RNA, enzymes, antibodies, and chemical compounds can be monitored and/or brought to reaction in the reaction chamber. Reaction chambers are known in which sensor systems of various kinds are situated on the bottom of the reaction chamber.

The invention relates to a method for monitoring changes and states in reaction chambers and to a supply unit that is required during experiments on cell cultures for introducing a liquid culture medium.

Known apparatus supply fresh culture medium or supply an active substance dissolved in this culture medium to the cells in a certain chronological sequence and remove used medium from the cell culture area. The supplied medium and the cell culture area must be protected from contamination by microorganisms and from excessive evaporation. These are important requirements for sensitive measurement of cellular reactions.

DE 19920811 describes an apparatus for performing experiments on cell cultures that are situated in a liquid culture medium. A separator is provided that can approach the cell culture located on a receptacle and that on top of the culture medium limits a reaction chamber. Provided inside the separator are one or a plurality of through-channels that open into the small-volume partial space of the receptacle. The convective mixing of the medium located in the reaction chamber and in the reservoir occurs in that a certain quantity of liquid culture medium is supplied via the through-channel to the reaction chamber and is evacuated again. The convective mixing occurs via the flow channel between separator and receptacle. Situated on the bottom of the separator is a profile with a convex curvature through which air and gas bubbles can escape.

Depending on ambient conditions, liquids can store or emit gases (gas exchange with the atmosphere), whereby the condition of saturation is always sought. Thus, there can be significant gas deposits, depending on temperature and pressure, among other things. Given a drop in pressure and an increase in temperature, a portion of the gas emitted to the environment can lead to the formation of bubbles. In closed systems, these bubbles can be transported and can lead to disturbances in chemical, physical, and biological processes, measurement results, or the metrology environment (e.g. damages to the carpet of cells or within a reaction chamber, inhibition of chemical reactions on surfaces because of accumulations of air bubbles).

It is a disadvantage of the prior art that gas bubbles can occur that negatively impact the cell culture or measurement by sensors.

Various methods and devices are known for avoiding or reducing disturbances caused by air bubbles.

Some of these systems (vacuum, heating, ultrasound,and so forth) can degas the liquid partially or nearly completely. However, care must be taken that no additional gas can be absorbed during further transport of the liquids (gas-impermeable transport containers/tubes/hoses). Furthermore, degassing can lead to changes in the properties of the liquid (e.g., denaturing of proteins by heating) and to effects on the sensors. For these reasons, the described degassing methods are not suitable for applications that are based on semi-open systems, that work with living (e.g. oxygen-consuming) cells, and/or that do not permit manipulation of the liquid.

Additional methods for air bubble suppression are e.g. so-called air bubble traps in the hose system. Air and gas bubbles rise in an area provided for this and are not further transported in the outflow. The disadvantage of this method is the additional dead volume (time delay/intermixing when media are changed) and where needed the required degassing site that comes into contact with the environment (e.g. possible contamination). Moreover, the system can only remove air bubbles that are disposed in the hose upstream of the trap (in the pump direction). Additional gas bubbles can form in the subsequent hose/line system.

### SUMMARY OF THE INVENTION

The object of the invention is to enable air bubble-free measurement in reaction chambers for monitoring changes and states in reaction chambers. Degassing is not to be used at all.

The method for monitoring changes and states in reaction chambers is characterized in that a fluid is drawn or pumped out of a reservoir and transported to a supply unit. The fluid drips or flows via a second through-channel (inlet channel) into a drip chamber so that air bubbles that are transported with the fluid remain on the fluid surface or escape into the environment immediately. Thus they cannot travel into the reaction chamber. The fluid forms a supply above a head and a reaction chamber. The height of the fluid surface and thus the supply volume is determined using a first through-channel (suction channel) and a fluid exchange occurs in the reaction chamber due to suctioning via the suction channel and the flowing of the fluid out of the drip chamber caused thereby.

In one exemplary embodiment the height of the fluid surface and thus the supply volume is determined using a third through-channel (emergency suction channel).

The change in the fluid or in a surface in the reaction chamber is initiated by living cells and/or chemical, biochemical, and/or immunological reactions, the fluid supply and draining occurring simultaneously or sequentially.

The reaction chamber can be changed using a lifting mechanism in the head carrier. The fluid in the drip chamber is thereby mixed with the fluid in the reaction chamber. In one exemplary embodiment, the liquid in the drip chamber is drawn into the reaction chamber (by suctioning the liquid out of the reaction chamber).

A membrane is arranged in the reaction chamber such that fluid does not flow directly into portions of the reaction chamber.

In the inventive supply unit for monitoring changes and states in reaction chambers, a first through-channel opening into the reaction chamber suctions a fluid. The inlet for the fluid occurs via a second through-channel above the fluid surface.

Sensor systems for detecting the change in the fluid are arranged in the reaction chamber and/or in the first through-channel.

In one exemplary embodiment, the head carrier comprises a head with a stock-shaped shaft and an enlargement for receiving the second through-channel.

In another exemplary embodiment, another enlargement for receiving a third through-channel, which as an emergency suction prevents an overflow, is situated above the enlargement and within the receptacle.

Another embodiment demonstrates that the second through-channel for supplying the fluid is arranged adjacent to the head carrier. The first through-channel is situated in the bottom of the reaction chamber.

The surface of the supply unit is provided with a hydrophobic and/or hydrophilic coating.

Degassers and bubble traps are unnecessary because of this structurally optimized supply unit, which supplies fresh reaction components and disposes of used reaction components and a new fluid guide.

Bubbles are captured directly at the flow-through head in the immediate vicinity of the reaction chamber and are prevented from being transported to the reaction chamber, whereby the physical, chemical, and biological properties of the fluid remain unchanged.

### BRIEF DESCRIPTION OF THE DRAWINGS

The exemplary embodiments are explained using the drawings.

FIG. 1—inventive supply unit with suction and inlet;

FIG. 2—inventive supply unit with suction, inlet, and emergency suction; and

FIG. 3—inventive supply unit in another embodiment.

### DETAILED DESCRIPTION OF THE DRAWINGS

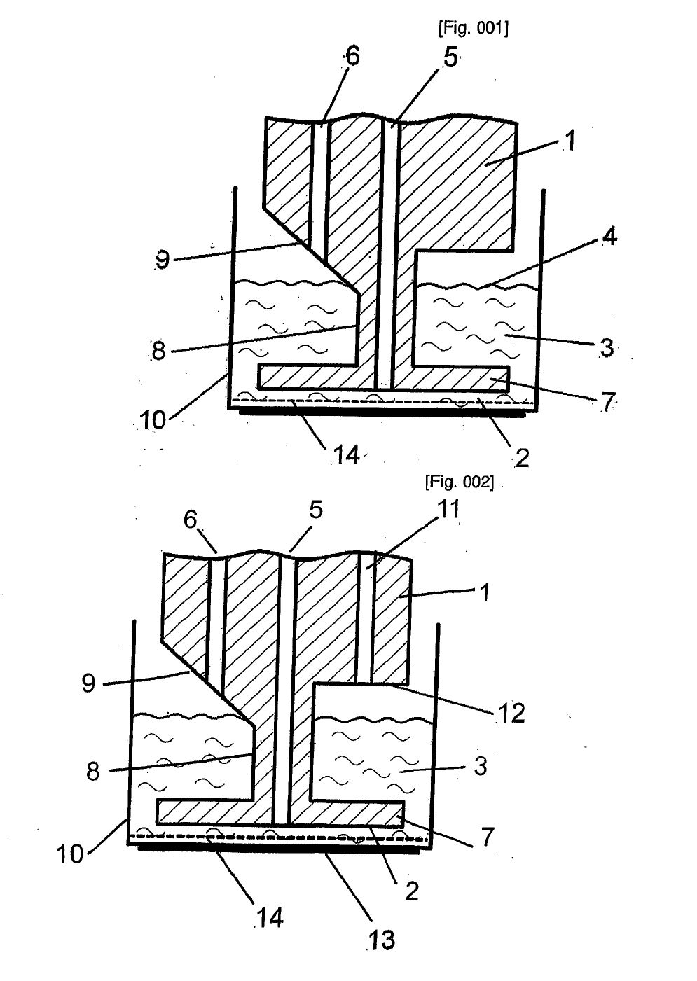

FIG. 1 depicts the inventive supply unit. Situated in a receptacle 10 is a head carrier 1 that limits the reaction chamber 2. Cells, cell components, DNA, RNA, enzymes, antibodies, and chemical compounds can be monitored and/or brought to reaction in the reaction chamber 2. Sensor systems 13 of various types can be disposed on the bottom of the reaction chamber 2 and/or in the first through-channel 5. These can be e.g. electrical, optical, and/or acoustic sensors. A membrane 14 in the reaction chamber 2 can retain e.g. suspension of cells or other mobile reaction components in the reaction chamber 2 or can prevent direct flow (shear forces) by adherent growing cells or reaction components on surfaces. FIG. 2 depicts how the inventive head carrier 1 with the first through-channel 5 as suction, the second through-channel 6 as inlet, and the third through-channel 11 as emergency suction can prevent an overflow.

The head carrier 1 has a head 7 with a connecting stock-shaped shaft 8. A first through-channel 5 that opens into the reaction chamber 2 acts to suction a fluid 3. The inlet occurs via a second through-channel 6 into a drip chamber above the fluid surface 4. This second through-channel 6 is situated in an enlargement 9 that is for instance semi-circular and beveled with respect to the stock-shaped shaft 8. Using this arrangement it is possible that no undesired bubbles or gases occur in the reaction chamber 2.

A certain liquid quantity of culture medium is supplied to the fluid 3, already present, via the second through-channel 6 from a reservoir via a hose and/or tube system. The fluid 3 drips or flows via the second through-channel 6 into the drip chamber. Air bubbles remain on the fluid surface 4 or escape immediately into the environment. The fluid is suctioned out of the reaction chamber 2 via the first through-channel 5. Thus unused bubble-free culture medium always travels into the reaction chamber 2 in that the fluid 3 flows out of the supply into the drip chamber. The fluid 3 in the drip chamber is drawn into the reaction chamber 2 by the suctioning of the liquid out of the reaction chamber 2. The height of the fluid surface 4 and thus the supply volume is determined using the first through-channel 5.

The height of the fluid surface 4 can also be determined using the third through-channel 11 as an emergency suction channel.

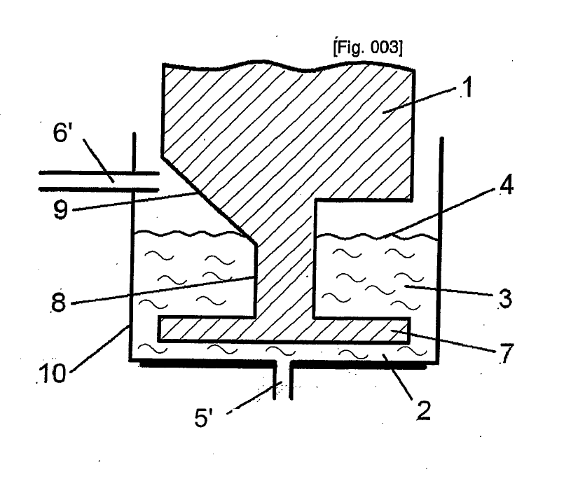

FIG. 3 illustrates another embodiment of the arrangements for the first and second through-channels 5′, 6′. Here, the second through-channel 6′ for supplying the fluid is arranged adjacent to the head carrier 1 and the first through-channel 5′ is arranged in the bottom of the reaction chamber 2. Theoretically other equivalent arrangements are also possible.

If the reaction chamber 2 is changed by a lifting mechanism of the head carrier 1, the fluid 3 in the drip chamber mixes with the fluid in the reaction chamber 2.

If the surface of the head carrier 1 and/or of the receptacle 10 is provided with a hydrophobic and/or hydrophilic coating, the properties of the fluids on the surfaces are affected such that air bubbles in the fluids can escape more simply and bubbles are captured directly at the through-flow head in the immediate vicinity of the reaction chamber 2 and are prevented from being transported to the reaction chamber 2.

The following are possible dimensions of the individual components:

Height of the reaction chamber: 200-500 μm

Height of the drip chamber: 0.5-3 mm

Height of the fluid surface: 1-5 mm

Aperture diameter for through-channels: 0.5-1 mm

The advantages of the new system are, first of all, the simple construction, and secondly, that no change occurs in the medium (liquid) since the gas portion in the fluid is not changed (degasser (heat, vacuum)). There is no ultrasound degassing or heating. The cells can be supplied adequately with gases (e.g. O₂).

Likewise, due to bubble-free and electrically certain coupled suction, as needed a reference electrode required for the measurement or other external sensors can be placed such that they themselves and/or their electrolytes do not have any undesired effect on the measurement.

Another advantage is that the reaction chamber can be minimized, the space for “passing through” air bubbles is no longer necessary. Likewise, reducing the size of the reaction chamber renders detectable changes in the fluid based on surface reactions and enables smaller volumes of test substances/test materials.

1.-16. (canceled)

17. method of providing an air bubble-free body of liquid having an exposed upper surface in a receptacle including a reaction chamber and allowing for monitoring of conditions in the liquid, comprising
inserting a member into the vessel, the member comprising a head portion forming an upper wall of the reaction chamber,
supplying the liquid to the receptacle by causing the liquid to drip or flow into the receptacle from a conduit at a location above the surface of any body of the liquid previously supplied to the vessel, whereby any air bubbles in the liquid being supplied separate from the liquid being supplied before the liquid being supplied enters said body of the liquid,
providing a conduit communicating with said body of the liquid from outside the receptacle,
applying suction to the conduit thereby to withdraw therethrough liquid from said body of the liquid, and
regulating height of said exposed surface by regulating said supplying and withdrawing of the liquid.

18. Method according to claim 17, wherein
both said conduits comprise channels formed through said member.

19. Method according to claim 18, further comprising
providing a third channel through the member, and
applying suction to the third channel thereby to withdraw therethrough liquid from said body of the liquid to assist in the regulating of the height of said exposed surface.

20. Method according to claim 17 or 18, wherein
the reaction chamber contains living cells, cell components, DNA, RNA, enzymes and/or antibodies and/or chemical, biochemical and/or immunological reactions are conducted therein.

21. Method according to claim 17 or 18, wherein
the supplying and the withdrawing of the liquid are continuous.

22. Method according to claim 17 or 18, wherein
the supplying and the withdrawing of the liquid are discontinuous.

23. Method according to claim 17 or 18,
wherein said exposed surface is above a first location of said reaction chamber wall,
and the method further comprising lifting said member thereby to raise said reaction chamber wall to a second location above the first location thereof, whereby at least a portion of said body of the liquid which had been above said reaction chamber wall is mixed into the reaction chamber liquid.

24. Method according to claim 17 or 18, further comprising
providing a membrane in the reaction chamber thereby to subdivide the reaction chamber into a portion into which the liquid being supplied directly flows and a portion into which the liquid being supplied does not directly flow.

25. Apparatus comprising a receptacle including a reaction chamber and means for supplying the receptacle with an air bubble-free body of liquid having an exposed upper surface, the apparatus being adapted for monitoring of condition, the means for supplying comprising
a member for insertion into the receptacle and comprising, at a lower extremity thereof, a head portion for forming an upper wall of the reaction chamber,
a first conduit for communicating between outside the receptacle and the body of liquid for suctioning liquid away from the body of liquid, and
a second conduit for communicating between outside the receptacle and the interior of the receptacle for supplying liquid to the receptacle, the second conduit having a lower extremity above a predetermined upper level of the body of liquid whereby liquid supplied through the second conduit drips or flows into the body of liquid from above the body of liquid during which dripping or flowing any air bubbles in the liquid being supplied separate from the liquid being supplied before the liquid being supplied enters the body of liquid.

26. Apparatus according to claim 25, wherein
the first and second conduits comprise first and second channels through the member.

27. Apparatus according to claim 26, wherein
the member further comprises a carrier portion from which the head portion depends, and
the second channel extends through the carrier portion and the head portion of the member thereby to communicate with the reaction chamber.

28. Apparatus according to claim 25, wherein
the receptacle comprises a bottom and the receptacle bottom forms a bottom of the reaction chamber, and
the first conduit comprises a channel through the bottom of the receptacle.

29. Apparatus according to claim 27, wherein
the carrier portion comprises a shaft portion through which the first channel passes and from which the head portion depends and, thereabove, a first enlarged portion of a greater lateral dimension than the shaft in a first lateral direction and the second channel passes through the first enlarged portion.

30. Apparatus according to claim 25, wherein
the second conduit comprises a channel through a side wall of the receptacle, said channel being adjacent the member.

31. Apparatus according to claim 29,
wherein the carrier portion further comprises a second enlarged portion of greater lateral dimension than the shaft in a second lateral direction, and
the apparatus further comprising a third channel, the third channel passing through the second enlarged portion for communicating with and suctioning away liquid from the body of liquid at a height of the body of liquid in the receptacle greater than a height of the body of liquid in the receptacle at which the first conduit first communicates with the body of liquid thereby to prevent the body of liquid from overflowing the receptacle.

32. Apparatus according to claim 25, further comprising
at least one of hydrophilic or hydrophobic coatings on surfaces of the apparatus.

33. Apparatus according to claim 25, further comprising
a sensor in the reaction chamber and/or the first conduit for monitoring conditions in the liquid.
